(19) United States
(12) Patent Application Publication
(10) Pub. No.: US 2021/0322619 A1
(43) Pub. Date: Oct. 21, 2021

(54) Anti-Viral Aerosolized Sterilization Technology

(71) Applicants: Eric Holohan, Huntington, NY (US); Barbara Dutton, Huntington Station, NY (US)

(72) Inventors: Eric Holohan, Huntington, NY (US); Barbara Dutton, Huntington Station, NY (US)

(21) Appl. No.: 17/234,447

(22) Filed: Apr. 19, 2021

Related U.S. Application Data
(60) Provisional application No. 63/012,805, filed on Apr. 20, 2020.

(51) Int. Cl. A61L 9/14; A61L 9/015

(52) U.S. Cl. CPC A61L 9/14; A61L 9/015; A61L 2209/134; A61L 2209/211; A61L 2209/212; A61L 2209/135

(57) ABSTRACT

An anti-viral system for generating and outputting an ozone-laden flow of air mixed with aerosolized hydrogen peroxide has housing including an air input port for receiving air and an output port for outputting the ozone-laden airflow mixed with aerosolized hydrogen peroxide. The system preferably includes an air movement device, an ozone supply, an aerosolization chamber and a fluid-flow path between the air input port and the output port, which includes the aerosolization chamber therebetween. A flow of air into the fluid-flow path is supplied with ozone from the ozone supply prior to reaching the aerosolization chamber, which aerosolizes the hydrogen peroxide within the ozone-laden air.

### CROSS-REFERENCE TO RELATED APPLICATIONS

This application derives the benefit of the filing date of U.S. Provisional Patent Application No. 63/012,805, filed Apr. 20, 2020. The contents of the provisional application are incorporated by reference in this application.

### BACKGROUND OF THE INVENTION

The invention relates to sanitization of closed spaces such as rooms, vehicle compartments, shared spaces, shared public spaces etc., which might be located on ships, at ports, on aircrafts, in airports, hotels, restaurants, trains, motor vehicles, houses, apartments, commercial areas, offices, stores, shops, warehouses, hospitals, ambulances, industrial premises, shipping containers, and refrigerated shipping containers, without limitation.

### SUMMARY OF THE INVENTION

The present invention was developed in an effort to limit or otherwise prevent transmission of coronaviruses such as COVID-19.

The present invention presents an anti-viral aerosolized sterilization technology (AVAST) system for the disinfection of domestic, commercial, medical, and public transportation spaces, which overcomes the shortcomings of prior art.

In an embodiment, the present invention provides a compact, preferably light weight anti-viral aerosolized sterilization technology (AVAST) system for disinfection of domestic, commercial, medical and transport spaces populated with people (hereinafter sometimes referred to herein as “peopled spaces”) that might unwittingly be exposed to and/or spread coronaviruses, such as COVID-19. The inventive system kills coronaviruses, such as COVID-19. Hence, by utilizing the inventive system in peopled spaces, the amount of active virus vastly decreases. And as the presence of coronaviruses, such as COVID-19, decreases, the risk of becoming infected, or infecting others in these peopled spaces decreases significantly-so much so that in peopled spaces protected by the inventive system, people may go about their business more or less without significant risk or infection. Concomitantly, the need for PPE (personal Protective Equipment) also is reduced.

The inventive system effectively disables or kills the coronaviruses, such as COVID-19, in reliance upon at least two (2) chemical materials, each of which display a track record with a very high kill rate for coronaviruses, such as COVID 19. The first chemical material is hydrogen peroxide (H₂O₂). Hydrogen peroxide has a long history and is very well documented for use as a disinfection agent; hydrogen peroxide is approved by the United States Food and Drug Administration (USFDA) for use as a disinfection agent. The second chemical material is ozone (O₃), which also is highly effective as an antipathogen. Ozone is known for use in the sterilization of drinking water systems and potable water, and understood by the inventors herein to be as effective when applied by the inventive system for killing coronaviruses, such as COVID-19.

The inventive anti-viral aerosolized sterilization technology (AVAST) system delivers an adjustable combination of these chemical methods which combines these two methods to produce a more effective system for the sanitization and disinfection/decontamination of areas where people could spread the virus, by disinfection of the air and the surfaces therein.

Third party testing has been carried out on several different pathogen challenge species. The test have shown excellent results where all pathogens were destroyed after the system was used, according to Table 1, and the related description describing how to utilize the inventive AVAST system optimally.

### BRIEF DESCRIPTION OF THE DRAWINGS

The invention will be described in conjunction with the following drawings in which like reference numerals designate like elements and wherein:

FIG. 1 is a sectional view of an embodiment of the anti-viral aerosolized sterilization technology (AVAST) system, constructed according to the inventive principles; and

FIG. 2 depicts a peopled environment in which the anti-viral aerosolized sterilization technology (AVAST) system of FIG. 1 is deployed, to deliver aerosolized hydrogen peroxide (H2O2) with ozone (O3) to a container that may contain people, i.e., a peopled space.

### DETAILED DESCRIPTION OF THE INVENTION

The following is a detailed description of example embodiments of the invention depicted in the accompanying drawings. The example embodiments are presented in such detail as to clearly communicate the invention and are designed to make such embodiments obvious to a person of ordinary skill in the art. However, the amount of detail offered is not intended to limit the anticipated variations of embodiments; on the contrary, the intention is to cover all modifications, equivalents, and alternatives falling within the spirit and scope of the present invention, as defined by the appended claims.

Embodiments of the present invention provide anti-viral aerosolized sterilization technology (AVAST) systems, methods and apparatus that reduce the number of active coronaviruses, such as COVID-19, capable of infecting people in a contained space (people space) through delivery, in a form of a mist, fog, or dry fog composed primarily of a microfine droplets of a solution of Hydrogen Peroxide with a concentration of less than 35%, but greater than 2.5%, of other sterilization agents or disinfectants, or disinfection agents, preferably ozone (O³).

Embodiments of the inventive anti-viral aerosolized sterilization technology (AVAST) system effectively spatially distribute the dry fog or mist. That is, the inventive system disperses the dry fog or mist in a flow of air driven by a fan and forced to exit the system. The system construction may widely disperse the dry fog or mist to fill an entire volume of a particular peopled space with a concentration sufficient to destroy the viruses present therein when they are contacted by the dry fog or mist. The system construction, however, also provides for modifying the process of sanitizing to localized portions of peopled spaces, as will be explained in greater detail below.

In some embodiments, the anti-viral aerosolized sterilization technology includes at least one nozzle, or ultrasonic device or resonator, which atomize or aerosolize the sterilization solution comprising the hydrogen peroxide, the first chemical material. The aerosolization nozzle or ultrasound device or resonator preferably is adapted to realize aerosolized hydrogen peroxide droplets sized substantially in range of between 100 nanometers and 10 microns (100 nm and 10 g), but most preferably, about 5 microns. In applicants' understanding, 1 milliliter (ml) of agent may be aerosolized to realize approximately 1.3 billion aerosolized droplets.

Preferably, the ultrasound device or resonator merely resonates at a fixed rate, whereby the actual air flow provided by the fan, which effectively determines the air flow past the ultrasound device or resonator, and, therefore, the volume of droplets of hydrogen peroxide, and the concentration of ozone in the outgoing air flow. The rate may be required to be adjusted due, for example, to humidity, extreme temperature, etc. The timer also may be adjusted to extend a time of treatment to reflect not only these conditions, but also a size of the closed room or space under treatment.

The smaller the droplet size, the less the mass and weight and, therefore, the greater the tendency to remain suspended in air. In applicants' understanding, droplets formed by the aerosolization average about 5 microns and accordingly remain airborne (once aerosolized) for up to 1 hour. In most cases, one hour of contact by the aerosolized droplets in a people space with air in which a coronavirus, such as COVID-19 is present therein is sufficient to destroy it.

In some embodiments, an ozone generator is arranged such that the air that is inducted into the anti-viral aerosolized sterilization technology unit passes through the ozone generator. Air is about 18% oxygen (O₂). Some part of the oxygen of the inducted air is converted into ozone (O₃) by the ozone generator, exits the ozone generator and then passes through the inventive system. As such, the inducted ozone (O₃) laden air is the air that is then processed to include the aerosolized first chemical material, e.g., hydrogen peroxide (H₂O₂), to add to the anti-virus effect of the dry fog or mist as expelled from the unit as a microfine aerosol.

Turning now to the drawing figures, the features of one or more exemplary embodiments of the invention will not be discussed, to highlight the inventive subject matter. The specific details are presented for exemplary purposes only, and not meant to be limiting.

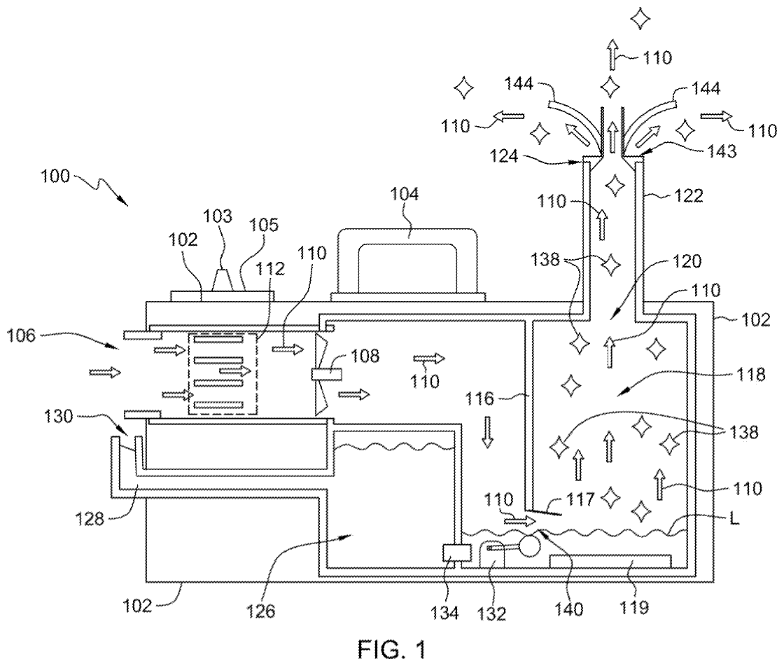

FIG. 1 which is a sectional view of an anti-viral aerosolized sterilization technology (AVAST) system 100. The AVAST system 100 comprises a housing 102, with an optional carrying handle 104. The housing 102 (and the handle 104) may be made of metal or plastic, such as aluminum, stainless steel, hardened plastics or other man-made polymer material, without limitation. An on/off switch 103 is used to activate and deactivate the system 100. Preferably, the switch 102 includes timer means 105, enabling the system to be activated for a specific period before it shuts itself off. For that matter, the timer means 105 might have a motion sensor (not expressly shown), whereby the AVAST system can be automatically activated/deactivator, if sensing a condition, such as a presence of people in a space for sterilization in which the AVAST system is deployed.

At what might be characterized as a front end of the system 100, is an air intake port 106. Air is input to the system at the air intake port 106, via a negative pressure created there by a fan or pump 108. The fan or pump creates an air flow into, through and out of the system's housing 102, in the direction of air flow arrows 110, in the embodiment shown. Proximate the air intake port 106, inside the housing 102, is an ozone generator 112. Incoming air passing through the ozone generator 112 is operated upon to convert some of the oxygen (O₂) within the air flow 110 to ozone (O₃), as a second chemical material to enhance the pathogen killing effect of the first chemical material, or hydrogen peroxide (H₂O₂). Air flow (see arrows 110) exiting the ozone generator 112, now mixed with Ozone (O₃), is drawn onward into the housing towards the fan or air pump 108, and pushed by the fan after passing the fan, as shown.

Those skilled in the art, upon reading this disclosure, will appreciate that features of the present invention may be used with desirable results on a number of other types, configurations and sizes of fans or air pumps 108. Moreover, the structural limitations and arrangements within the housing may be modified, without deviating from the scope and spirit of the invention, as long as the arrangement ensures that air flowing in is processed to include ozone (O₃) and atomized, or aerosolized hydrogen peroxide. For example, the fan or pump 108, may be arranged to precede the ozone generator 112 (in the airflow direction; see air flow arrows 110). For that matter, the ozone generator 112 may be replaced with an ozone supply, which injects measured amounts of ozone into the air flow passing through the ozone supply/generator, without deviating from the scope and spirit of the invention.

The ozone (O₃) laden air flow is directed, in reliance upon one or more baffles 116, to an aerosolization chamber 118, via a flow path from the ozone generator 112, and fan or pump 108 (depending one the physical arrangement), within the internal volume to the aerosolization chamber 118, where the H₂O₂ is aerosolized into the air flow 110 containing the ozone-laden air. The now aerosolized air flow, with the aerosolized hydrogen peroxide, then exits the housing 102 via output port 120. Preferably, a stovepipe-like tube or fluid communication device 122, or other means for facilitating fluid communication of the ozone-laden air with aerosolized H₂O₂ droplets 138, is attached to or integrally extends from output port 120.

The tube 122 essentially extends a “reach” of the output port to an output end 124 of tube 122, as shown.

Preferably, the output end or port 124 of stovepipe-like device 122 comprises a director diffuser 143, as shown. The director diffuser 143 is located at the output port 124 of the stovepipe-like tube 122, preferably to direct the ozone-laden air flow 110 mixed with the aerosolized hydrogen peroxide in an axially symmetrical direction, in reliance upon wings or directors 144. The wings or directors allow for directing the air flow, including upwards as shown, to ensure a directional dispersal of the materials The director diffuser 143 may be attached to the tube 122 proximate its output port by friction fit, snap on device, or any connection device know to persons of ordinary skill in the art.

The inventive system includes a fluid reservoir 126 for holding hydrogen peroxide (H₂O₂), to be aerosolized in the aerosolization chamber 118 thereby becoming part of the ozone-laden air flow. A fluid communication channel 128, with a fill port 130 for receiving a supply of hydrogen peroxide liquid, leads to the fluid reservoir 126. Hence, preferably liquid hydrogen peroxide solution or other sterilization agents or disinfectants are delivered to the reservoir 126 through the fill port 130. In the embodiment shown, a float switch 132, which operates with a valve or pump 134, may be controlled to maintain a level, and therefore an amount of hydrogen peroxide (H₂O₂), or other sterilization agents or disinfectants to be aerosolized, to be supplied from the fluid reservoir into the aerosolization chamber 118.

The amount of the hydrogen peroxide (H₂O₂) aerosolized into and mixed with the ozone laden air flow, per unit volume, is influenced by the level of fluid in the aerosolization chamber 118, and an ultrasound (u/s) transducer 119. For purposes of illustration the aerosolized hydrogen peroxide droplets, or other sterilization agents or disinfectants mixed in the ozone laden air in the aerosolization chamber 138, are identified as stars 138.

In the embodiment depicted, the air and Ozone (O₃) (ozone-laden air flow 110) travel past the fan or pump 108 and descend along the baffle wall 116 and enter the aerosolization chamber 118 through a gap 140 between an end 117 of the baffle wall 116, and a surface of the liquid hydrogen peroxide at level L, as shown. A size of the gap 140 is determined by a level of hydrogen peroxide fluid in the chamber 138, and therefore, the level of the liquid leaving the liquid reservoir 126.

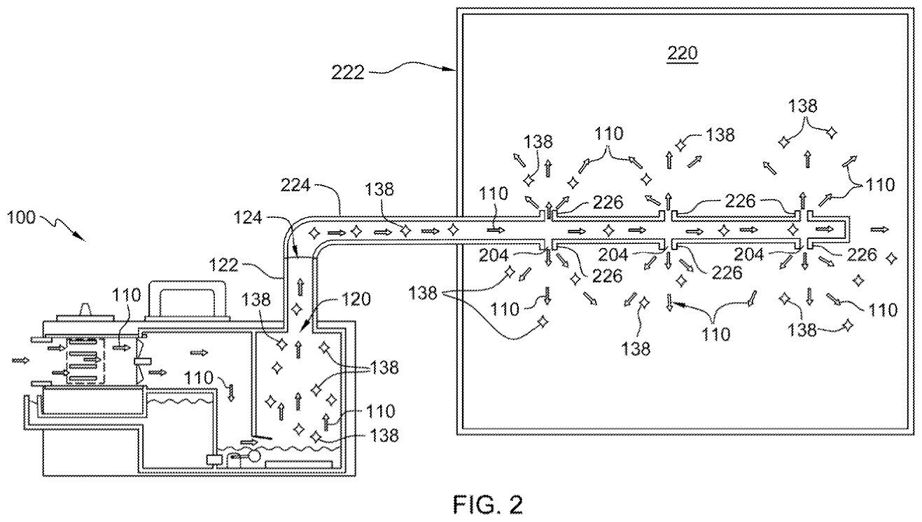

FIG. 2 presents an application of the inventive system 100 in an effort to sanitize an exemplary closed space or volume 220, enclosed by walls 222. The enclosed space or volume is representative of any room, vehicle compartment, shared space, shared public space, etc., which might be located on ships, at ports, on aircrafts, in airports, hotels, restaurants, trains, motor vehicles, houses, apartments, commercial areas, offices, stores, shops, warehouses, hospitals, ambulances, industrial premises, school classrooms, and shipping containers, and refrigerated shipping containers in which the system is operation to maintain substantially pathogen-free.

The output port 124 of the stovepipe-like device 122 of the AVAST system 100 is connected via a pipeline or fluid communication tube 224, to closed space or volume 220, as shown. This may be an area where clothing, tools or other goods also can be sterilized with the mixture of aerosolized hydrogen peroxide 138 in the ozone-laden air flow 110. The pipeline or fluid communication tube 224 extends into the space 220 (as shown) and preferably includes a plurality of fluid distribution ports or vent 226. In some embodiments, the dispersal of the mixture of aerosolized hydrogen peroxide 138 in the ozone-laden air flow 110 may alternatively be accelerated, or better directionally delivered by use of fluid delivery nozzles, which in that case could be positioned at the location of vents 226. Please note that the pipeline or fluid communication tube 224, while depicted as a single contiguous pipe, may be separated into sections, for convenience, without deviating from the scope or spirit of the invention.

While manufacturing and other considerations may impact the physical dimensions, the inventors have discovered the following design attributes provide desirable results for general use. In general, the overall weight of the AVAST system unit should not exceed 10 kg for a hand portable unit, where the unit is filled with the Hydrogen Peroxide solution or other sterilization agents or disinfectants and ready for use. Larger AVAST systems could be fitted with rolling wheels to permit the use of larger fluid reservoirs to allow less frequent filling, pragmatic when deployed in larger spaces to be sterilized.

Pursuant to some embodiments, including the embodiments depicted in FIGS. 1 and 2, the AVAST system is capable of treating about 20,000 cubic feet per hour, to achieve the desired effective concentration of hydrogen peroxide solution or other sterilization agents or disinfectants or disinfection agents, in the ozone-laden air (flow), for a given closed volume or space to be treated. Please note that as the AVAST system 100 pulls air into the housing 102 for processing before directional output into the closed space or volume for treatment (e.g., 102). Hence, depending on the time in which the AVAST system is actively processing (during the treatment time period), the concentration of hydrogen peroxide solution or other sterilization agents or disinfectants or disinfection agents, in the ozone-laden air (flow), increases the amount of droplets, and concentration of ozone, in the ozone-laden air, as the air flow is recycled.

Given that an exemplary space of volume for treatment, such as space or volume 220 (FIG. 2), comprises a standard 8 foot ceiling, a square area footprint (where the volume is length in feet, by width in feet, by the 8 foot height), of such closed volume or space is 2500 square feet (such as by a square of about 50 feet by 50 feet). Where the exemplary space or volume has a 5000 square foot footprint, or a 10,000 foot footprint, the AVAST system would need to operate for 2 hours, and 4 hours respectively. The fixed dosage rate for the FIGS. 1 and 2 AVAST system may distribute greater than 1 liter of H₂O₂ per hour. Preferably, the rate of ozone supplied to realize ozone laden air into which the greater than 1 liter per hour of hydrogen peroxide is mixed with 10,000 milligrams of ozone per hour.

These are fixed rates and provide a very heavy saturation for disinfection purposes. Adjusting the fan adjusts the rate, for example, if there appears to be making the area around the output port damp, the fan speed may be increased, which lessens the concentration in the outgoing air flow. Pursuant to some embodiments, to achieve these desired concentration of hydrogen peroxide solution or other sterilization agents or disinfectants or disinfection agents in the closed volume or space, it a simple table showing the amount of time of operation that is required to achieve this concentration (see Table 1, which reflects an 8 foot ceiling height in the room or space defined by the floor footprint in square feet and meters).

The timer switch or controller 103, 105 may be set to this 1 hour duration once the volume of the closed space is calculated.

TABLE 1

| | |
|---|---|
| Room Area | Treatment Time |

| Sq. Feet | Sq. Meters | Minutes |
|---|---|---|
| 50 | 5 | 1 |
| 100 | 9 | 2 |
| 200 | 19 | 4 |
| 300 | 28 | 5 |
| 400 | 37 | 7 |
| 500 | 46 | 8 |
| 600 | 56 | 10 |
| 700 | 65 | 11 |
| 800 | 74 | 13 |
| 900 | 84 | 15 |
| 1000 | 93 | 17 |
| 1500 | 139 | 26 |
| 2000 | 186 | 41 |
| 2500 | 232 | 60 |

As will be evident to persons skilled in the art, the foregoing detailed description and figures are presented as examples of the invention, and that variations are contemplated that do not depart from the fair scope of the teachings and descriptions set forth in this disclosure. The foregoing is not intended to limit what has been invented, except to the extent that the following claims so limit that.

1. An anti-viral system for generating and outputting an ozone-laden flow of air mixed with aerosolized hydrogen peroxide, the anti-viral system comprising:
a system housing including an air input port for receiving air and an output port for outputting the ozone-laden airflow mixed with aerosolized hydrogen peroxide;
an air movement device;
an ozone supply;
an aerosolization chamber; and
a fluid-flow path between the air input port and the output port, which includes the aerosolization chamber therebetween;
wherein the air movement device generates a flow of air into the fluid-flow path, via the air input port that is supplied with ozone from the ozone supply prior to reaching the aerosolization chamber, which aerosolizes the hydrogen peroxide within the ozone-laden air and output via the output port as the ozone-laden airflow mixed with aerosolized hydrogen peroxide.

2. The anti-viral system of claim 1, wherein the system housing is made of a lightweight material comprising metal or man-made material such as plastic.

3. The anti-viral system of claim 1, wherein the air movement device is a fan and the ozone supply comprises a nozzle or like device connected by fluid communication means to a container containing ozone, and a regulator for regulating an amount of ozone injected into the air flow per unit time during intended anti-viral system operation.

4. The anti-viral system of claim 1, wherein the ozone supply is an ozone-generating device arranged within the flow of air into the device to convert oxygen therein to ozone.

5. The anti-viral system of claim 4, wherein air movement devices is arranged at a first position within the flow of air as to draw the flow of air through the ozone-generating device.

6. The anti-viral system of claim 4, wherein air movement devices is arranged at a second position within the flow of air as to push the flow of air through the ozone-generating device.

7. The anti-viral system of claim 1, wherein the aerosolization chamber is arranged within the fluid flow path after, in a flow of air direction, the ozone generating device.

8. The anti-viral system of claim 7, wherein the aerosolization chamber includes an ultrasonic aerosolization device to aerosolize hydrogen peroxide into the ozone-laden flow of air.

9. The anti-viral system of claim 8, further comprising a controller for controlling an amount of aerosolization per unit volume in the flow of air through the aerosolization chamber.

10. The anti-viral system of claim 1, further comprising a fluid reservoir for receiving and storing a supply of hydrogen peroxide for aerosolization.

11. The anti-viral system of claim 10, further comprising means enabling fluid communication of hydrogen peroxide within the fluid reservoir to the aerosolization chamber.

12. The anti-viral system of claim 11, wherein the means enabling fluid communication of hydrogen peroxide within the fluid reservoir to the aerosolization chamber includes means for regulating an amount of hydrogen peroxide as a function of time or weight.

13. The anti-viral system of claim 1, further comprising a first tube or other fluid communication device detachably attached to the output port, that distributes the ozone-laden flow of air mixed with aerosolized hydrogen peroxide.

14. The anti-viral system of claim 13, further comprising a directional diffuser connected to an output port of the first tube or other fluid communication device, for directionally distributing the ozone-laden flow of air mixed with aerosolized hydrogen peroxide.

15. The anti-viral system of claim 13, further comprising at least one nozzle, for controlling dispersement of an ozone-laden flow of air mixed with aerosolized hydrogen peroxide into the space to be treated.

16. The anti-viral system of claim 1, further comprising a secondary fluid communication pipe, connected to the first tube or other fluid communication device adapted to deliver the ozone-laden flow of air mixed with aerosolized hydrogen peroxide from outside a closed space or chamber to be treated into the closed space or chamber.

17. The anti-viral system of claim 1, further comprising a wheel assembly connected to the housing to enable easy movement.

18. The anti-viral system of claim 1, further comprising a timer switch or controller for activating and deactivating the system, the ozone generator or the aerosolization.

19. The anti-viral system of claim 1, further comprising a float switch or liquid level controller, valve or liquid pump to maintain a desired flow of hydrogen peroxide into the aerosolization chamber.

20. The anti-viral system of claim 1, wherein a size of the aerosolized hydrogen peroxide droplets is in a range between 100 nanometers and 10 microns (100 nm and 10μ).
